(19) United States
(12) Patent Application Publication
(10) Pub. No.: US 2018/0112210 A1
(43) Pub. Date: Apr. 26, 2018

(54) BROAD-SPECTRUM SEROLOGICAL DIAGNOSTICS AND USES THEREOF

(71) Applicant: UNIVERSITY OF HOUSTON SYSTEM, Huoston, TX (US)

(72) Inventors: Navin Varadarajan, Huoston, TX (US); Jay R. Adolacion, Huoston, TX (US); Richard C. Willson, Huoston, TX (US)

(73) Assignee: University of Houston System, Huoston, TX (US)

(21) Appl. No.: 15/571,220

(22) PCT Filed: May 23, 2016
(86) PCT No.: PCT/US2016/033722

§ 371 Date: Nov. 1, 2017

Related U.S. Application Data
(60) Provisional application No. 62/165,396, filed on May 22, 2015.

(51) Int. Cl. C12N 15/10

(52) U.S. Cl. CPC C12N 15/1062

(57) ABSTRACT

In some embodiments, the present disclosure pertains to method of screening a biological sample for a plurality of diseases. In some embodiments, such a method comprises obtaining a biological sample from a subject in need thereof. In some embodiments, the biological sample comprises a plurality of biomarkers. In some embodiments, each of the plurality of biomarkers is specific for at least one disease. In some embodiments, the method comprises contacting the biological sample with a display library of peptides. In some embodiments, each peptide in the library may have a unique amino acid sequence. In some embodiments, each of the peptides is physically linked to a nucleic acid sequence that identifies of encodes the peptide. In some embodiments, at least one of the peptides is capable of binding to at least one of the biomarkers in the biological sample. In some embodiments, the method comprises separating the bound peptide particles from the unbound peptide particle. In some embodiments, the method comprises eluting the bound peptide particles from the bound state. In some embodiments, the method comprises determining the identity of the nucleic acid sequences physically linked to the bound peptide particles. In some embodiments, the method comprises comparing the sequences thus obtained to a database of sequences representing a plurality of biomarkers of diseases.

### CROSS REFERENCE TO RELATED APPLICATIONS

This application claims priority to U.S. Provisional Patent Application No. 62/165,396, filed in the United States Patent and Trademark Office on May 22, 2015, the entirety of which is hereby incorporated by reference.

### FIELD OF INVENTION

The invention pertains to tools and methods for the early detection, diagnosis, monitoring, and/or treatment of a disease or a condition, and biomarkers and arrays for use in the same.

### BACKGROUND

Early detection of a condition can have a significant impact on the outcome of a disease, and yet, for most conditions, no single test exists that can detect disease before the appearance of major symptoms. Existing diagnostic assays for specific conditions are limited to a specific disease or diagnosis. Moreover, monitoring health over a period of time is cost and time-prohibitive for currently available diagnostic assays. This is especially true in resource-poor settings. Therefore, there exists a need to develop diagnostic assays and methods for identifying a wide-variety of conditions at an early stage.

### BRIEF SUMMARY

In some embodiments, the present disclosure pertains to method of screening a biological sample for a plurality of diseases. In some embodiments, such a method comprises obtaining a biological sample from a subject in need thereof. In some embodiments, the biological sample comprises a plurality of biomarkers. In some embodiments, each of the plurality of biomarkers is specific for at least one disease. In some embodiments, the method comprises contacting the biological sample with a display library of peptides. In some embodiments, each peptide in the library may have a unique amino acid sequence. In some embodiments, each of the peptides is physically linked to a nucleic acid sequence that identifies or encodes the peptide. In some embodiments, at least one of the peptides is capable of binding to at least one of the biomarkers in the biological sample. In some embodiments, the method comprises separating the bound peptide particles from the unbound peptide particle. In some embodiments, the method comprises eluting the bound peptide particles from the bound state. In some embodiments, the method comprises sequencing the eluted peptides. In some embodiments, the method comprises determining the identity of the nucleic acid sequences physically linked to the bound peptide particles. In some embodiments, the method comprises comparing the nucleic acid sequences thus identified to a database of sequences representing a plurality of biomarkers of diseases.

In some embodiments, the present disclosure pertains to a method for monitoring response to treatment of on-going disease in a subject in need thereof. In some embodiments, the method comprises obtaining a biological sample from the subject. In some embodiments, the method comprises contacting the biological sample with a display library of peptides. In some embodiments, each peptide in the library may have a unique amino acid sequence. In some embodiments, each of the peptides is physically linked to a nucleic acid sequence that encodes the peptide. In some embodiments, at least one of the peptides is capable of binding to at least one biomarker in the biological sample. In some, embodiments, the method diagnoses a known disease with known biomarkers of the specific disease. In such embodiments, the number of different types of peptides in the library is from 2 to 100,000 or from 2 to ten million. In some embodiments, the method diagnoses unknown disease with unknown biomarkers. In such embodiments, the number of different types of peptides in the library is from one hundred to one trillion, or from one thousand to one billion. In some embodiments, the number of different types of peptides in the library is from ten thousand to one billion. In some embodiments, the number of different types of peptides in the library is from 2 to 500. In some embodiments, the number of different types of peptides in the library is from 100 to one 100,000. In some embodiments, the method comprises separating the bound peptide particles from the unbound peptide particles.

In some embodiments, the method comprises eluting the bound peptide particles from the bound state. In some embodiments, the method comprises sequencing the eluted peptides. In some embodiments, the method comprises determining the identity of the nucleic acid sequences physically linked to the bound peptide particles. In some embodiments, the method comprises comparing the nucleic acid sequences thus identified to a database of sequences representing a plurality of biomarkers for the disease. In some embodiments, the presence or absence of the at least one biomarker is indicative of the response to the treatment.

In some embodiments the present disclosure pertains to a method of establishing an immunosignature specific for diagnosing a disease in a subject in need thereof. In some embodiments, such a method comprises obtaining a biological sample from the subject. In some embodiments, the method comprises contacting the biological sample to an immobilized peptide microarray. In some embodiments, each of the immobilized peptide in the microarray has a unique amino acid sequence. In some embodiments, each of the peptides is physically linked to a nucleic acid sequence that encodes for the peptide. In some embodiments, each of the immobilized peptides is capable of binding to at least one immunodominant epitope binding antibody specific for the disease present in the biological sample.

In some embodiments the method comprises detecting binding of the immobilized peptides in the microarray to the antibody in the biological sample to obtain a binding profile of the biological sample. In some embodiments the method comprises separating the bound peptides from the unbound peptides on the immobilized microarray display library. In some embodiments the method comprises eluting the bound peptides from the bound state. In some embodiments, the method comprises determining the identity of the nucleic acid sequences physically linked to the bound peptide particles. In some embodiments, the method comprises comparing the nucleic acid sequences thus identified to a database of sequences representing a plurality of diseases to diagnose the disease. In some embodiments, the binding of the at least one antibody to a plurality of different peptides in the peptide array comprises an immunosignature specific for the disease.

### BRIEF DESCRIPTION OF THE FIGURES

FIG. 1 is a representative schematic for capturing and screening antibody biomarkers of disease. IgGs from patient sera are isolated by immobilization onto a surface (for example, using protein G). In a process called panning, the purified IgGs are interrogated with a phage library displaying peptides that can in part or in whole correspond to disease epitopes. Bound phage are eluted off and propagated to filter down and generate a more specific phage library. After several rounds of panning, the genetic information encoded in the enriched phage library is retrieve using high-throughput sequencing to identify the peptide sequences that map out disease epitopes.

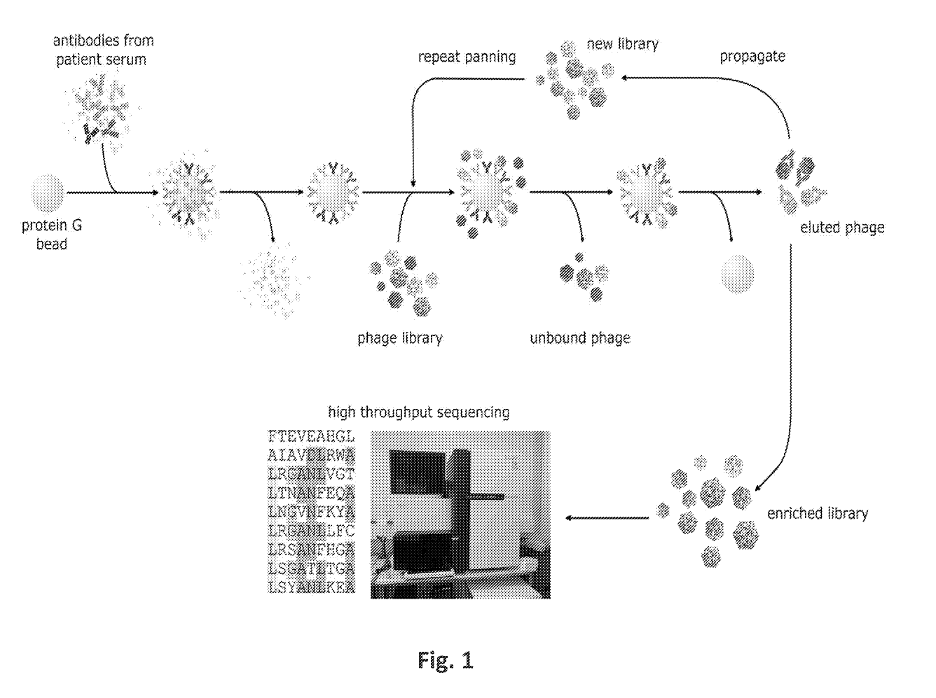

FIG. 2 represents a protocol for panning antibodies immobilized onto a surface using MS2 VLPs. With MS2 VLPs, IgGs captured onto a well surface is panned against MS2 VLP library (FIG. 2). Propagation is carried out by a combination of RT-PCR and of the nucleic acid sequence corresponding to the phage-displayed peptide and performing traditional cloning techniques to generate a new library.

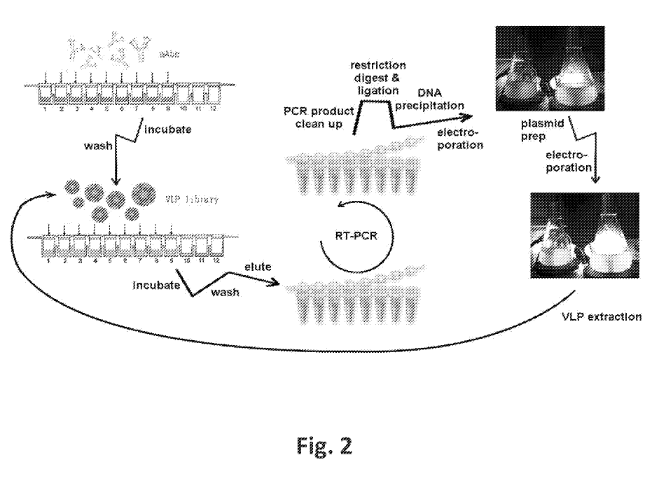

FIG. 3 shows a mixture of 1,000 ng FLAG MS2 VLPs and 10,000 ng WT MS2 VLPs in a 10 μL final volume panned against surface-bound anti-FLAG mAbs. Surface-bound anti-FLAG mAbs were prepared by incubating well surfaces with 500 ng, 250 ng, or 125 ng anti-FLAG mAbs in 10 μL. A no-MS2 RT and a no-MS2 PCR run as well as the starting FLAG MS2 VLP spike-in solution (1,000 ng in 10 μL final volume) offered for panning are included as controls. The spike-in solution was analyzed in a separate RT-qPCR experiment.

FIG. 4 shows a mixture of 100 ng FLAG MS2 VLPs and 10,000 ng WT MS2 VLPs in a 10 μL final volume panned against surface-bound anti-FLAG mAbs. Surface-bound anti-FLAG mAbs were prepared by incubating well surfaces with 500 ng, 250 ng, or 125 ng anti-FLAG mAbs in 10 μL. A no-MS2 RT and a no-MS2 PCR run as well as the starting FLAG MS2 VLP spike-in solution (100 ng in 10 μL final volume) offered for panning are included as controls. The spike-in solution was analyzed in a separate RT-qPCR experiment.

FIG. 5 shows a mixture of 10 ng FLAG MS2 VLPs and 10,000 ng WT MS2 VLPs in a 10 μL final volume panned against surface-bound anti-FLAG mAbs. Surface-bound anti-FLAG mAbs were prepared by incubating well surfaces with 500 ng, 250 ng, or 125 ng anti-FLAG mAbs in 10 μL. A no-MS2 RT and a no-MS2 PCR run as well as the starting FLAG MS2 VLP spike-in solution (10 ng in 10 μL final volume) offered for panning are included as controls. The spike-in solution was analyzed in a separate RT-qPCR experiment.

### DETAILED DESCRIPTION

It is to be understood that both the foregoing general description and the following detailed description are exemplary and explanatory only, and are not restrictive of the invention, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that comprise more than one unit unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Unless defined otherwise, all technical and scientific terms used herein have the meaning commonly understood by a person skilled in the art to which the invention belongs. The following references provide one of skill with a general definition of many of the terms used in this disclosure: Singleton et al., Dictionary of Microbiology and Molecular Biology (2^{nd} ed. 1994); The Cambridge Dictionary of Science and Technology (Walker ed., 1988); The Glossary of Genetics, 5^{th} ed., R. Reigers et al. (eds.), Springer Verlag (1991); and Hale & Marham, The Harper Collins Dictionary of Biology (1991).

As used herein, the term “sample” refers to a biological material which is isolated from its natural environment and contains at least one antibody. A sample according to the methods described herein, may consist of purified or isolated antibody, or it may comprise a biological sample such as a tissue sample, a biological fluid sample, or a cell sample comprising an antibody. A biological fluid includes, but is not limited to, blood, plasma, sputum, urine, cerebrospinal fluid, lavages, and leukophoresis samples, for example.

As used herein, the term “display library” refers to a library comprising a plurality of peptides. In some embodiments, the display library may be representative of the entire human genome. In some embodiments, the display library comprises of a plurality of peptides representative of a plurality of biomarkers, where at least one biomarker is specific for at least one disease, and where the display library comprises biomarkers for a plurality of diseases. In some embodiments, the display library comprises a plurality of peptides representative of a plurality of biomarkers, where the biomarkers are specific for a disease. In some embodiments, the display library comprises peptides derived from a plurality of diseases. In some embodiments, the peptides are specific for biomarkers, antibodies, and/or proteins specific for a particular disease. In some embodiments, the disease is an autoimmune disease. In some embodiments, the disease is a cancer. In some embodiments, the disease is a neurological disease. In some embodiments, the peptides are specific for pathogens that are displayed on the surface of a virus or cell e.g., bacteriophage, yeast, or bacteria.

As used herein, the term “antibody-peptide complex” refers to a complex formed when an antibody recognizes an epitope on a peptide and binds to the epitope under low or normal stringent conditions. It will be appreciated that an antibody-peptide complex can dissociate under high stringent conditions, such as low or high pH, or high temperatures. As used herein “bound library elements” or “bound peptide particles” refers to a complex formed when a biomarker in the biological sample recognizes an epitope on a peptide and binds to the epitope under low or normal stringent conditions. It will be appreciated that the bound library elements or the bound peptide particles can dissociate under high stringent conditions, such as low or high pH, or high temperatures.

As used herein, the term “reaction sample” refers to a sample that, at a minimum, comprises a phage display library, for example, the phage display library described herein. The reaction sample can also comprise additional buffers, salts, osmotic agents, etc., to facilitate the formation of complexes between the peptides in the phage display library when the reaction sample is contacted with a biological sample comprising an antibody, protein, or a biomarker specific for a disease or a condition. A “biological sample” as that term is used herein refers to a fluid or tissue sample derived from a subject that comprises or is suspected of comprising at least one antibody, protein, or a biomarker specific for a disease or a condition.

A biological sample can be obtained from any organ or tissue in the individual to be tested, provided that the biological sample comprises, or is suspected of comprising, an antibody. Typically the biological sample will comprise a blood sample; however other biological samples are contemplated herein, for example cerebrospinal fluid.

In some embodiments, a biological sample is treated to remove cells or other biological particulates. Methods for removing cells from a blood or other biological sample are well known in the art and can include e.g., centrifugation, ultrafiltration, immune selection, or sedimentation etc. Antibodies or biomarkers can be detected from a biological sample or a sample that has been treated as known, to those of skill in the art. Some non-limiting examples of biological samples include a blood sample, a urine sample, a semen sample, a lymphatic fluid sample, a cerebrospinal fluid sample, a plasma sample, a serum sample, a pus sample, an amniotic fluid sample, a bodily fluid sample, a stool sample, a biopsy sample, a needle aspiration biopsy sample, a swab sample, a mouthwash sample, a cancer sample, a tumor sample, a tissue sample, a cell sample, a synovial fluid sample, or a combination of such samples. For the methods described herein, it is preferred that a biological sample is from whole blood, plasma, cerebral spinal fluid, serum, and/or urine. In one embodiment, the biological sample is cerebrospinal fluid.

In some embodiments, samples can be obtained from an individual with a disease or pathological condition. In some embodiments, samples can be obtained from an individual undergoing treatment for a disease or a pathological condition. In some embodiments, samples can be obtained from healthy individuals.

In some embodiments, the present disclosure relates to a serological diagnostic approach to identify a wide variety of diseases and conditions in subjects in need thereof. In some embodiments, the diseases and conditions include but are not limited to tropical diseases, other infectious diseases, neurological diseases, diverse forms of cancers, autoimmune disorders, and allergies. In some embodiments, the method(s) disclosed herein also affords access to diagnostic reagents for diseases that have an immunological aspect, either as consequence or cause. In some embodiments, the methods disclosed herein pertain to monitoring of on-going treatment of a disease or a condition. In some embodiments, the methods disclosed herein may be used to predict prognosis of a disease or a condition being treated.

In some embodiments, the present disclosure pertains to method of screening a biological sample for a plurality of diseases. In some embodiments, such a method comprises obtaining a biological sample from a subject in need thereof. In some embodiments, the biological sample comprises a plurality of biomarkers. In some embodiments, the pluralities of biomarkers are specific for at least one disease. In some embodiments, each of the plurality of biomarkers is specific for at least one disease. In some embodiments, the method comprises contacting the biological sample with a display library of peptides. In some embodiments, each peptide in the library may have a unique amino acid sequence. In some embodiments, each of the peptides is physically linked to a nucleic acid sequence that encodes for the peptide. In some embodiments, at least one of the peptides is capable of binding to at least one of the pluralities of biomarkers in the biological sample. In some embodiments, the method comprises separating the bound peptide particles from the unbound peptide particle. In some embodiments, the method comprises eluting the bound peptide particles from the bound state. In some embodiments, the method comprises determining the identity of the nucleic acid sequences encoding the bound peptide particles. In some embodiments, the method comprises comparing the nucleic acid sequences of the bound peptide particles thus identified to a database of sequences representing a plurality of biomarkers of diseases.

In some embodiments, the disease is selected from breast cancer, prostate cancer, bladder cancer, soft tissue sarcoma, lymphomas, esophageal cancer, uterine cancer, bone cancer, adrenal gland cancer, lung cancer, thyroid cancer, colon cancer, glioma; liver cancer, pancreatic cancer, renal cancer, cervical cancer, testicular cancer, head and neck cancer, ovarian cancer, neuroblastoma, melanoma, acute lymphocytic leukemia, chronic lymphocytic leukemia, acute myeloid leukemia, T cell leukemias, and B cell leukemias.

In some embodiments, the disease is a neurological disease. In some embodiments, the neurological disorder is selected from the group consisting of Alzheimer's disease (AD), pre-AD, Parkinson's disease, multiple sclerosis, fronto-temporal dementia, bipolar disorder, or schizophrenia.

In some embodiments, the disease is an autoimmune disease. In some embodiments, the autoimmune disease is selected from a group consisting of rheumatoid arthritis, systemic lupus erythematosus (SLE), Hashimoto's disease, Graves' disease, Addison's disease, and Sjögren's syndrome.

In some embodiments, the method further comprises the step of immobilizing the plurality of biomarkers in the biological sample on to a support.

In some embodiments, the at least one biological sample comprises blood, dry blood, serum, plasma, saliva sample, mucosal swab, biopsy, a tissue, skin, hair, feces, urine, synovial fluid, cerebrospinal fluid, interstitial fluid, and pleural fluid.

In some embodiments, the pluralities of biomarkers comprise immunoglobulin isotypes. In some embodiments, the immunoglobulin isotypes comprise IgG, IgA, IgM, T cells, MHC, immune-fractionated antibodies, or combinations thereof.

In some embodiments, the method further comprises affinity purifying at least one immunoglobulin isotype from the biological sample. In some embodiments, the affinity-purified immunoglobulin isotype is contacted with an immobilized display library of peptide particles. In some embodiments, the pluralities of biomarkers comprise a plurality of proteins, where each of the plurality of proteins is specific for at least one specific disease. In some embodiments, the pluralities of proteins are specific for at least one disease.

In some embodiments, the display library of peptide particles comprises of peptides displayed on a phage, spore, yeast, cell, ribosome, DNA, RNA, a colloids, a surface, or a label. In some embodiments, the peptides are selected from the group consisting of a random peptide, a cyclic peptide, an enzyme-modified peptide, a chemically-modified peptide, a glycosylated peptide, an entire protein, a random peptide, a moving-window peptide along a protein sequence, or a peptide representing an epitope specific for a disease marker, or a plurality of peptides representing immunodominant epitopes specific for a disease marker. In some embodiments, the peptides are chemically or enzymatically modified. In some embodiments, the display library of peptide particles is configured as a lateral-flow or as a microtiter plate assay.

In some embodiments, the method further comprises labeling sequences or chemicals associated with the displayed peptides. In some embodiments, the labeling utilizes RNA, DNA, small organic molecules, fluorescence or metals. In some embodiments, the display library of peptides comprises peptides encoded in the human genome. In some embodiments, the display library of peptides comprises peptides representative of biomarkers for a plurality of cancers. In some embodiments, the display library of peptides comprises a plurality of peptides representative of biomarkers for a single disease.

In some embodiments, the plurality of peptides in the library is from 2 to 100,000 or from 2 to ten million. In some embodiments, the plurality of peptides in the library is from one hundred to one trillion, or from one thousand to one billion. In some embodiments, the plurality of peptides in the library is from ten thousand to one billion. In some embodiments, the plurality of peptides in the library is from 2 to 500. In some embodiments, the plurality of peptides in the library is from 100 to one 100,000. In some embodiments, the method comprises separating the bound peptide particles from the unbound peptide particles.

In some embodiments, the present disclosure pertains to a method for monitoring response to treatment of on-going disease in a subject in need thereof. In some embodiments, the method comprises obtaining a biological sample from the subject. In some embodiments, the method comprises contacting the biological sample with a display library of peptides. In some embodiments, each peptide in the library may have a unique amino acid sequence. In some embodiments, each of the peptides is physically linked to a nucleic acid sequence that encodes for and identifies the peptide. In some embodiments, at least one of the peptides in the display library is capable of binding to at least one biomarker in the biological sample. In some embodiments, the method comprises separating the bound peptide particles from the unbound peptide particles. In some embodiments, the method comprises eluting the bound peptide particles from the bound state. In some embodiments, the method comprises determining the identity of the nucleic acid sequences physically linked to the bound peptide particles. In some embodiments, the method comprises comparing the nucleic acid sequences of the bound peptide particles thus identified, to a database of sequences representing a plurality of biomarkers for the disease. In some embodiments, the presence or absence of the at least one biomarker is indicative of the response to the treatment.

In some embodiments, the peptide display library of the present disclosure may be structured to detect with high sensitivity a pattern of binding of a small quantity of a biological sample to a plurality of peptides in the array. In some embodiments, the present disclosure provides a method of detecting, processing, analyzing, and correlating the pattern of binding of the biological sample to the plurality of peptides with a condition or a disease state. In some embodiments, the present disclosure produces an “Immunosignature,” which is associated with a disease, a condition, an early onset of disease or condition, or evaluating efficacy of a treatment of a disease or condition in a subject. Immunosignaturing detects and partitions an antibody response into a coherent set of signals. A coherent set of signals from an immunosignature obtained with the arrays and methods disclosed herein may provide a robust and comprehensive method for the diagnosis and early detection of various conditions, including cancer, inflammation, autoimmune disease, infection, and other physiological conditions. Immunosignaturing is distinct from and an alternative to traditional, individual protein or genetic biomarkers for the diagnosis of various conditions. A coherent set of signals from an Immunosignature obtained with arrays and methods of the present disclosure may be used as an effective method of preventive care, health monitoring, diagnosis, and as a method of treatment.

In some embodiments the present disclosure pertains to a method of establishing an immunosignature specific for diagnosing a disease in a subject in need thereof. In some embodiments, such a method comprises obtaining a biological sample from the subject. In some embodiments, the method comprises contacting the biological sample to a peptide display library. In some embodiments, each of the peptides in display library has a unique amino acid sequence. In some embodiments, each of the peptides in the display library is capable of binding to at least one immunodominant epitope binding antibody specific for the disease present in the biological sample.

In some embodiments the method comprises detecting binding of the peptides in the display library to the biological sample to obtain a binding profile of the biological sample. In some embodiments, the binding profile comprises the aggregate effect of a plurality of antibodies in the biological sample bound to the different peptides.

In some embodiments the method comprises separating the antibody-bound peptides from the unbound peptides of the display library. In some embodiments the method comprises eluting the bound peptides from the bound state. In some embodiments, the method comprises determining the identity of the nucleic acid sequences physically linked to the bound peptide particles. In some embodiments, the method comprises comparing the nucleic acid sequence of the bound peptides thus identified, to a database of sequences representing a plurality of diseases to diagnose the disease. In some embodiments, the sequences thus obtained comprises the immunosignature specific for the diagnosed disease.

In some embodiments, the methods and arrays of the present disclosure are used to diagnose a disease in a subject in need thereof. In some embodiments, such a method comprises obtaining a biological sample from the subject. In some embodiments, the method comprises contacting the biological sample to a peptide display library. In some embodiments, the peptide display library comprises different peptides capable of binding to at least one antibody present in the biological sample. In some embodiments, the binding of the antibody to a plurality of different peptides in the peptide display library comprises an immunosignature specific for the disease. In some embodiments, the method comprises diagnosing the disease based on the immunosignature.

In some embodiments, the methods of the present disclosure pertain to assessing the efficacy of a treatment in a subject in need thereof.

The methods of the present disclosure may use any biological sample obtained from a subject. For example, the biological sample may be blood, dry blood, serum, plasma, saliva sample, mucosal swab, biopsy, a tissue, skin, hair, cerebrospinal fluid sample, feces, or urine. In some embodiments, the biological sample is a blood sample that is contacted to a peptide array of peptide sequences. Additionally, the biological sample utilized by the methods and arrays of the present disclosure may include interstitial fluid, synovial fluid, and needle aspirates. In some embodiments, the biological sample comprises a plurality of disease markers. In some embodiments the plurality of disease markers comprise isolated antibody subclass, IgG, IgM, IgA, T cells, MHC, immune-fractionated antibodies, a protein, a fusion protein, an allergen, or a pathogen.

The present disclosure provides methods for the association of a biological sample obtained from a subject, such as a blood, a dry blood, a serum, a plasma sample, a saliva sample, a check swab, a biopsy, a tissue, a skin, a hair, a cerebrospinal fluid sample, feces, or a urine sample, to the state of health of the subject. In some embodiments, the biological sample is a blood sample that is contacted to a peptide array of peptide sequences

In some embodiments, a subject can, for example, use a “fingerstick”, or “fingerprick” to draw a small quantity of blood and add it to a surface, such as a filter paper or other absorbent source, or in a vial or container and optionally dried. A biological sample obtained, for example, from a drop of a subject's blood and placed on a filter paper can be directly mailed to a provider of the methods of the present disclosure without a processing of the sample. A biological sample provided by a subject can be concentrated or dilute.

A biological sample may be derived from a plurality of sources within a subject's body and a biological sample can be collected from a subject in a plurality of different circumstances. A biological sample may be collected, for example, during a routine medical consultation, such as a blood draw during an annual physical examination. A biological sample may be collected during the course of a non-routine consultation, for example, a biological sample may be collected during the course of a biopsy. A biological sample can be obtained from any organ or tissue in the individual to be tested, provided that the biological sample comprises, or is suspected of comprising, an antibody. Typically the biological sample will comprise a blood sample, however other biological samples are contemplated herein, for example, cerebrospinal fluid.

In some embodiments, a biological sample is treated to remove cells or other biological particulates. Methods for removing cells from a blood or other biological sample are well known in the art and can include e.g., centrifugation, ultrafiltration, immune selection, or sedimentation etc. Antibodies can be detected from a biological sample or a sample that has been treated as described above or as known, to those of skill in the art. Some non-limiting examples of biological samples include a blood sample, a urine sample, a semen sample, a lymphatic fluid sample, a cerebrospinal fluid sample, a plasma sample, a serum sample, a pus sample, an amniotic fluid sample, a bodily fluid sample, a stool sample, a biopsy sample, a needle aspiration biopsy sample, a swab sample, a mouthwash sample, a cancer sample, a tumor sample, a tissue sample, a cell sample, a synovial fluid sample, or a combination of such samples. For the methods described herein, it is preferred that a biological sample is from whole blood, plasma, cerebral spinal fluid, serum, and/or urine. In one embodiment, the biological sample is cerebrospinal fluid.

In some embodiments, the display library of peptides comprises displaying proteins or peptides derived from both natural and non-natural amino acids. In some embodiments, the proteins or peptides are displayed on a phage, bacterial, spore, yeast, cell, ribosome, particle, surface, label. General methods for producing a phage display library are known to those of skill in the art and/or are described in e.g., Larman et al. (201 1) Nature Biotechnology 29(6):535-541, which is incorporated herein by reference in its entirety. Contemplated herein are phage display libraries, where each library comprises a plurality of particles, where each particle displays peptides representative at least one biomarker specific for a disease, or a condition. In one embodiment, it is contemplated herein that the plurality of peptides will represent a substantially complete set of peptides representing the human genome. In one embodiment, the phage display library comprises a substantially complete set of peptides representing biomarkers for all known cancers. Similarly, phage display libraries comprising a substantially complete set of peptides representing biomarkers for all known neurological disease. Additionally, in an embodiment, the display library comprises a substantially complete set of peptides representative of biomarkers for auto-immune diseases. In some embodiments, the display library comprises a substantially complete set of peptides representative of biomarkers for a disease.

In some embodiments, the peptides comprise random peptides, cyclic, enzyme-modified, chemically-modified, glycosylated, entire proteins, and random peptides, moving-window peptides along a protein sequence, epitopes, and immunodominant epitopes. In some embodiments, the peptides/proteins may be modified by chemical or enzymatic action before testing for binding. In some embodiments, the modification comprises citrullination or modification through click chemistry. In some embodiments, the identified peptides/proteins are used in other formats, e.g., in binding assays in formats such as lateral-flow or microtiter plate assays. In some embodiments, the method comprises encoding or labeling sequences or chemicals associated with the displayed proteins/peptides. In some embodiments, the encoding or labeling utilizes RNA, DNA, small organic molecules, fluorescence or metals.

In some embodiments, the methods of the present disclosure comprise utilizing bacteriophage MS2 virus-like particle (MS2-VLP) affinity selection platform coupled with deep-sequencing. In some embodiments, the methods and arrays of the present disclosure utilize published literature and WWW curations of the immunodominant proteins or epitopes of a plurality of pathogens, to identify peptides for each biomarker specific for disease or condition to be displayed on the surface of the MS2-VLP platform. In some embodiments, the library of MS2-VLPs displaying pathogen-associated peptides is used for affinity selection with a biological sample from a subject and then deep-sequenced to identify peptides recognized by antibodies or biomarkers present in the biological sample. MS2 phage VLPs are unique in that they encapsidate their own coding RNA, providing the genotype- phenotype linkage to allow iterative rounds of affinity selection against a monoclonal antibody of interest or a polyclonal mixture of antibodies (i.e., patient serum, needle aspirates, ‘pinch’ biopsies). This linkage also can be achieved by any of the classical display technologies.

In some embodiments, the present disclosure pertains to a method of contacting the biological sample with the display library of peptides. In some embodiments, the method further comprises contacting, competing, blocking, passivating, and/or normal background elements. In some embodiments, the elements include but are not limited normal serum, blocking antibodies, detergents, polyethylene glycol, competitors, species variant protein, species variant peptides, or allergen-free variants.

In some embodiments the method comprises detecting binding of the peptides in the peptide display library to the biological sample.

In some embodiments, it is desirable to separate the library elements bound to the biomarkers in the biological sample, from those not bound to the biological sample or the free library elements. In some embodiments, the separation may be achieved by washing, wicking, or electrophoretic, magnetic or flotation forces. In one embodiment, bound biomarkers in the biological sample are immobilized on a solid support o permit one to separate out the unbound library elements. For example, where the reaction mixture comprises an antibody bound to a phage particle, essentially any method that permits one to specifically immobilize IgA, or IgG subclasses (e.g., IgG4) can be used to immobilize antibodies from the sample, including antibodies bound to one or more bacteriophage. In some embodiments, Protein A, Protein G or a combination thereof is/are used to immobilize the antibody to permit removal of unbound phage. Such methods are known to those of ordinary skill in the art and as such are not described in detail herein.

In some embodiments, the peptide or protein used to immobilize antibodies from the reaction mixture can be attached to a solid support,such as, for example, magnetic beads (e.g., micron-sized magnetic beads), sepharose beads, agarose beads, a nitrocellulose membrane, a nylon membrane, a column chromatography matrix, a high performance liquid chromatography (HPLC) matrix or a fast performance liquid chromatography (FPLC) matrix for purification. For example, the reaction mixture comprising bacteriophage and antibodies can be contacted with magnetic; beads coated with Protein A and/or Protein G. The Protein A and G will bind to antibodies in the mixture and immobilize them the beads. This process also immobilizes any phage particles bound by the antibodies in one embodiment, a magnet can be used to separate the immobilized phage from unbound phage.

In some embodiments, the methods of the present disclosure comprise elution of the bound library elements from the bound state. In some embodiments, the elution may be achieved by methods including but not limited to acid elution, temperature elution, base elution, solvent elution, chaotrope or protease action, use of a redox agent, the action of a competitor, partitioning, electrophoresis, or the passage of time for dissociation.

In some embodiments, the method comprises determining the identity of the nucleic acid sequences physically linked to the bound peptide particles. The peptides eluted from in the bound library element/biomarker complexes can be identified using e.g., PCR. In one embodiment, the nucleic acids from the lysed phage are subjected to an amplification reaction, such as a PCR reaction. For example, the nucleic acids encoding a phage-displayed peptide comprise a common adapter sequence for PCR amplification. In such embodiments, a PCR primer is designed to bind to the common adapter sequence for amplification of the DNA corresponding to a phage-displayed peptide.

In some embodiments, the methods disclosed herein comprise isolating or preparing a label associated with the bound peptide/protein of the peptide display library for analysis. In some embodiments, the step of isolating or preparing a label for analysis includes but is not limited to acid, base, chaotrope, or mechanical lysis, the action of a protease, solvent, or detergent, or adsorption, and/or nucleic acid labeling, amplification or reverse transcription.

In some embodiments, the method further comprises the step of detection of the label. In some embodiments, the detection is performed by methods including but not limited to sequencing, PCR, RPA, NASBA, LAMP, mass-tag PCR, in vitro transcription, in vitro translation, in vitro transcription and translation, in vitro translation to produce a reporter protein, hybridization, growing phage, yeast or bacteria, molecular beacon detection, imaging, enzymatic action, or the use of chromogenic, fluorogenic, or luminogenic substrates.

As used herein, “label” or “detectable label” refers to any atom or molecule which can be used to provide a detectable (preferably quantifiable) signal, and which can be operatively linked to a polynucleotide, such as a PCR primer. Labels may provide signals detectable by fluorescence, radioactivity, colorimetry, X-ray diffraction or absorption, magnetism, enzymatic activity, mass spectrometry, binding affinity, hybridization radiofrequency, nanocrystals and the like. A primer of the present invention may be labeled so that the amplification reaction product may be “detected” by “detecting” the detectable label.

In certain embodiments, the detection of a phage-displayed peptide comprises high throughput detection of a plurality of peptides simultaneously, or near simultaneously. In some embodiments, the high-throughput systems use methods similar to DNA sequencing techniques.

In some embodiments, the methods of the present disclosure comprise comparing the nucleic acid sequence of the eluted bound library elements to a database of sequences representing biomarkers of a plurality of diseases. In some embodiments, the database of sequences comprises of human exome sequence, genome sequences, GenBank, table of epitopes, database of immunodominant epitopes, a listing of associations between label/encoding/associated nucleic acid sequences and displayed proteins or peptides, a database of pathogen-derived sequences, a database of autoimmune-based disease associated sequences, or a listing of autoimmune-based disease-associated sequences.

In some embodiments, the methods of the present disclosure comprise performing an informatics step for the interpretation of results. In some embodiments, the interpretation of results comprises comparison of bound peptide/protein sequences to human exome sequences, comparison to genome sequences, comparison to GenBank, look up in a table of epitopes, comparison to a database of immunodominant epitopes, interpretation of amplification results to a listing of associations between label/encoding/associated nucleic acid sequences and displayed proteins or peptides, look-up against a database of pathogen-derived sequences, look-up against database of autoimmune-based disease-associated sequences, or comparison to a listing of autoimmune-based disease-associated sequences.

Previously, antibody samples have been tested against synthetic peptides, but the methods and compositions disclosed herein radically increases the size of the library which can be screened, and the immunodominant epitope approach has not been tested at all.

In some embodiments of the present disclosure, the methods disclosed herein utilize the MS2-VLP affinity selection coupled with deep-sequencing. In some embodiments, the methods and arrays disclosed herein are able to distinguish between pluralities of biomarkers in a biological sample in a single test. An MS2-VLP library displaying a number of biomarker-associated peptides would be affinity-selected against biological samples derived from a subject, the selected library would be deep-sequenced, and resultant data would be analyzed to identify the biomarker-associated peptides against which the subject had antibodies.

A balance needs to be struck between the breadth of biomarkers included versus cost and complexity. As more peptides are included in the library, the more oligonucleotide chips must be purchased and more sequencing may be required. For instance, including a large number of biomarkers but each represented by very few peptides may limit the ability of this serological diagnostic to identify false negatives. However, including fewer total biomarkers but each biomarker represented by a large number of peptides may decrease false negatives, but limit the ability of this serological diagnostic to be generalized to all/many patients.

It is important to note that there is no inherent technical limitation on the number of pathogens/epitopes that could be represented by this library, and chip costs are not prohibitive—$10,000-20,000 would enable a huge number of diseases to be represented. Alternately, it is possible to offer randomized peptides displayed on the phage to ‘select’ for phage particles binding to the biological sample of interest. This would be an unbiased and more comprehensive approach and is very inexpensive to implement, requires no prior knowledge of the underlying disease, and is scalable to billions of unique peptide sequences with very low costs associated with genetic construction of the library. High-throughput sequencing after limited screening of 1-2 rounds will identify the candidate peptides that can facilitate diagnosis.

The methods of the present disclosure may be used, for example, to diagnose, monitor, characterize, and guide treatment of a plurality of different conditions of a subject.

A subject may be a human, a guinea pig, a dog, a cat, a horse, a mouse, a rabbit, and various other animals. A subject may be of any age, for example, a subject may be an infant, a toddler, a child, a pre-adolescent, an adolescent, an adult, or an elderly individual.

A condition of a subject can correspond to a disease or a healthy condition. In some embodiments, a condition of a subject is a healthy condition, and a method of the present disclosure monitors the healthy condition. In some embodiments, a condition of a subject is a disease condition, and a method of the present disclosure is used to diagnose/monitor a state and/or the progression of the condition. A method of the present disclosure may also be used in the prevention of a condition. In some embodiments, a method of the invention is used in conjunction with a prophylactic treatment.

The methods disclosed herein importantly detect and monitor a variety of diseases and/or conditions simultaneously. For example, the methods disclosed herein are capable of simultaneously detecting inflammatory conditions, cancer, autoimmune diseases, neurological diseases, and pathogenic infection on the same array. Accordingly, only one display library may be sufficient to detect wide spectra of diseases and conditions. Alternatively, the same biological sample may be panned against a plurality of peptide display libraries, where each peptide display library represents different disease subsets. For example, in one embodiment, the biological sample may be panned against or contacted with a first peptide display library comprising peptides substantially representing biomarkers of all known cancers. In some embodiments, the method further comprises of contacting the same biological sample with a second peptide display library comprising peptides substantially representing biomarkers of all known autoimmune diseases.

In some embodiments the methods disclosed herein are used to probe for immune responses associated with targets of the immune response. In some embodiments, the targets of immune response include but are not limited to pathogens, proteins expressed in cancer, cancer fusion proteins, human autoimmune target proteins, food and environmental allergens, modifications such as citrullination, or penicillin.

In some embodiments, the methods and compositions pertain probing the efficacy of vaccination and the breadth and coverage of the underlying immune response. In some embodiments, the efficacy of the vaccination and the breadth of coverage of the underlying immune response are evaluated after tumor peptide vaccination. In some embodiments, the target proteins probed for are associated with autoimmune diseases including but not limited to rheumatoid arthritis, Crohn's disease, multiple sclerosis, SLE, lupus, celiac disease, type 1 diabetes, Grave's disease, Hashimoto's disease, myasthenia gravis, narcolepsy, psoriasis. In some embodiments, the target proteins probed for are associated with diseases including but not limited to Parkinson's disease, Alzheimer's disease, Myalgic Encephalomyelitis/Chronic Fatigue Syndrome, and Gulf War Syndrome. In some embodiments, the allergens probed for include but are not limited to soya, milk, peanuts, tree nuts, eggs, wheat, pollen, plants, citrus, fish, shellfish, dust mites, digestive enzymes of insects or arachnids, feathers, cat dander, dog dander.

### Application and Advantages

The present disclosure pertains to a method of screening a biological sample for a plurality of diseases using peptide display libraries representing biomarkers for a plurality of diseases in a single library. The present disclosure is advantageous over existing platforms for a variety of reasons. Firstly, the methods of the present disclosure do not require immobilizing arrays of particles bearing the peptides. The biological sample to be screened can be contacted with the peptide display library in solution. This greatly reduces the cost of making the display libraries. Secondly, the display library of the present disclosure comprises of a plurality of particles for screening a plurality of diseases. Further, each particle in the display library displays multiple peptides that are specific for a biomarker for a disease. This allows for an increased sensitivity of detection. Finally, the methods disclosed herein have the potential to screen multiple disease conditions because each display library has a plurality of particles, where each particle displays peptides specific for at least one disease biomarker. Furthermore, the same biological sample obtained from a subject undergoing screening or monitoring can be used to serially contact multiple such libraries, where each library is representative of a plurality of disease conditions. For the methods and systems disclosed detection the methods and systems can be utilized without immobilizing the peptide library because of a lack of

The methods disclosed herein comprise using a diagnostic platform known as immunosignaturing that uses random peptides printed onto a microarray resulting in a pattern that can be correlated with a specific disease condition. A great diversity of random peptide sequences can be achieved by phage display. This diversity is crucial to finding peptide sequences recognized by antibodies that can be used as disease markers. This approach has the benefit of identifying a plurality of diseases from a single sample obtained from a single subject. Further, the present disclosure also utilizes pre-identified immunodominant epitopes for higher quality probing of the response, and uses thereof.

The methods of the present disclosure may have several applications. For example, the methods disclosed herein may be utilized to capture IgG/IgM/IgA directly from a biological sample obtained from a subject onto beads/plates, pan using an unbiased library of phage particles and use high throughput screening (HTS) to look for immunosignatures. In some embodiments, the phage particles display linear/cyclic peptide. In some embodiments, the phage particles display small scaffold proteins like cysteine knots. In some embodiments, the phage particles display higher affinity/conformational epitopes. In some embodiments, the phage particles display predetermined preselected sequences based on known immunodominant epitopes or alternately just overlapping peptide pools for pathogens of interest

In some embodiments, the present disclosure pertains to a method of assessing the efficacy of vaccination by quantifying immunosignatures. In some embodiments, the present disclosure pertains to a method of prescreening to reduce the pathogen space for which a subject needs to be tested. In some embodiments, the methods disclosed herein utilize high throughput screening (HTS) and BLAST to determine likely pathogens that can then be tested using standard approaches. This can also be employed in the case of poorly-characterized illnesses like Gulf War Syndrome.

In some embodiments, the present disclosure pertains to a method of identifying biomarkers of disease. For example, the methods disclosed herein may be uses to test the hypothesis that Parkinson's/Alzheimer's produce a consistent humoral response against misfolded proteins

In some embodiments, the methods of the present disclosure are useful for diagnosing or screening a plurality of cancers. In some embodiments, the present disclosure pertains to using the immunosignature (IS) to stratify cancer subtypes with the knowledge that they are likely to elicit different humoral responses. In some embodiments, the present disclosure relates to a method of probing for proteins that are overexpressed or mutated in certain forms of cancers. In some embodiments, the present disclosure relates to a method of probing for unique fusion proteins produced in certain forms of cancer.

In some embodiments, the present disclosure pertains to quantifying the efficacy of oncolytic or antibody-based immunotherapy to stratify responders and non-responders. In some embodiments, the method comprises assessing for a shift in immunosignature pre and post administration of the therapy to quantify response to therapy or make treatment decisions.

### Additional Embodiments

Reference will now be made to more specific embodiments of the present disclosure and experimental results that provide support for such embodiments. However, Applicants note that the disclosure below is for illustrative purposes and is not intended to limit the scope of the claimed subject matter in any way.

### EXAMPLE 1

A library of cyclic peptides displayed or MS2 virus-like particles is contacted with enriched IgM of a human patient, each VLP containing an RNA sequence encoding the peptide which is displayed on the VLP. After extensive washing, the bound peptide-displaying VLPs are eluted from the immobilized IgM molecules. The nucleic acids contained therein are isolated and sequenced, and the sequences corresponding to displayed peptides are searched against a database of immunodominant proteins of 50 pathogens. The presence in the patient's body of antibodies recognizing immunodominant epitopes of Ehrlichia chaffeensis is interpreted as evidence of infection of the patient by this organism.

### EXAMPLE 2

A library of M13 phage particles displaying on their gp3 tail fibers cyclized peptides derived from the sequences of immunodominant protein epitopes of 100 different pathogens, is contacted with IgA isolated from stool of a human patient with gastrointestinal symptoms. After washing with detergent-containing solutions for selectivity, Bound phage are recovered and the peptide-encoding portions of the phage genomes are amplified by PCR and sequenced and recovery of phage sequences corresponding to sequences of immunodominant epitopes of Cryptosporidium is interpreted as evidence of Cryptosporidium infection of that patient.

### EXAMPLE 3

A library of 20 types of bacterial spores each of the 20 types displaying a peptide corresponding to the fusion junction of a cancer-associated protein, is contacted with Protein A—purified IgG antibodies from a human immobilized on one micron magnetic particles and allowed to bind with gentle mixing. Magnetic particles are retained by a magnet and the unbound spores are poured off, and then washed with fresh buffer three times. Spores are then eluted from the immobilized IgG population using glycine buffer at pH 2.5, spore nucleic acids are isolated and sequenced, and the greater-than-random recovery of a particular NPM-ALK fusion sequence is taken as evidence that the human has the form of cancer known as ALCL.

### EXAMPLE 4

A library of 20 types of M13 phage particles is prepared, in four groups of five. Each group of five types displays peptides derived from immunodominant epitopes from one of the following pathogens: Rickettsia prowazekii, Crimean-Congo Hemorrhagic Fever virus, dengue virus, and Chikungunya virus. Each phage also contains in its genome, a common DNA sequence amplifiable by a known pair of PCR primers. Within these sequences, each group of five types contains a distinctive sequence for which an individual Taqman PCR probe has been prepared. The phage library is contacted with IgG isolated from the blood of a human patient with fever of unknown origin. After washing with detergent-containing solutions for selectivity, bound phage are eluted and introduced into a TaqMan PCR reaction. TaqMan signaling of the presence of the label DNA sequence associated with the phage displaying Chikungunya epitopes is interpreted as evidence t at the patient is infected with Chikungunya virus.

### EXAMPLE 5

A library of 10 types of MS2 VLP particles is prepared, in two groups of five. Each group of five types displays peptides derived from immunodominant epitopes from dengue or Chikungunya viruses. Each VLP also contains a DNA sequence which contains a T7 promoter upstream of a gene encoding either green or red fluorescent protein. All five types displaying peptides derived from Chikungunya contain DNA encoding GFP, and all five types displaying peptides from dengue virus encode DNA containing RFP. The phage library is contacted with IgG isolated from the blood of a human patient with fever of unknown origin. After washing with detergent-containing solutions for selectivity, bound phage are eluted with acidic glycine solution, neutralized, and introduced into an in vitro transcription-translation reaction mixture. After 15 minutes, the presence of fluorescence consistent with GFP wavelengths is taken as evidence of infection of the patient by dengue virus

### EXAMPLE 6

A library of cyclic peptides displayed on MS2 virus-like particles is contacted with particle-immobilized enriched IgG of a human patient infected with Ehrlichia chaffeensis, each VLP containing an RNA sequence encoding the peptide which is displayed on the VLP. After extensive washing, the bound peptide-displaying VLPs are eluted from the immobilized IgM or IgG molecules. The nucleic acids contained therein are isolated and sequenced, and the sequences of the peptides displayed on the VLPs which were bound are inferred. These peptides are sequenced and used as capture agents in a lateral-flow assay for the serological diagnosis of Ehrlichia chaffeensis infection.

### EXAMPLE 7

A library of cyclic peptides displayed on MS2 virus-like particles is contacted with particle-immobilized enriched antibodies of a human patient showing food allergies, with each VLP containing an RNA sequence encoding the peptide which is displayed on the VLP. After extensive washing, the bound peptide-displaying VLPs are eluted from the immobilized IgM molecules. The nucleic acids contained therein are isolated and sequenced, and the sequences of the peptides displayed on the VLPs which were bound are inferred. The sequences of these peptides are used to infer the foods to which the patient is allergic.

### EXAMPLE 8

A library of 10 types of MS2-VLP particles is prepared, in two groups of five. Each group of five types displays peptides derived from immunodominant epitopes from dengue or Chikungunya viruses. Each VLP also contains a DNA sequence which contains a T7 promoter upstream of gene encoding a peptide defining the smaller portion 9f a split GFP reporter. The phage library is contacted with IgG isolated from the blood of a human patient with fever of unknown origin. After washing with detergent-containing solutions for selectivity, bound phage are eluted with acidic glycine solution, neutralized, and introduced into an in vitro transcription-translation reaction mixture containing a protein or peptide constituting the balance of GFP, complimentary to the peptide encoded in the DNA in the VLP. After 15 minutes, the presence of fluorescence consistent with GFP wavelengths is taken as evidence of infection of the patient by dengue or Chikungunya virus.

### EXAMPLE 9

A library of bifunctionalized M13 phage with immunodominant peptides specific to a certain type of cancer is displayed on pVII and a biotin tag on pill is chemically conjugated with a chromogenic reporter. This library is incubated with IgGs pulled down from human sera using magnetic protein G beads. The beads are washed for unbound phage and then treated with the corresponding chromogenic substrate. A detectable color change is taken to be evidence of the presence of that

### EXAMPLE 10

A library of bifunctionalized M13 phage with immunodominant peptides specific to a certain type of cancer is displayed on pVII and a biotin tag on pill is incubated with human sera. The antibody-phage pairs formed are captured using magnetic Strepavidin beads. These beads are then placed in contact with anti-human secondary antibodies and then any unbound fraction washed off. The corresponding signal produced by these antibodies serves as marker for that cancer.

### Example 11

A library of bifunctionalized M13 phage is constructed for detecting different cancer types. Each cancer is represented by a subset of immunodominant epitopes displayed on pVII at one end of the phage and a unique purification tag on pill at the other end. This library is incubated with IgGs captured from human sera. The unbound fraction is washed off and the bound fraction is then eluted off. This eluent is passed through an LFA strip with several test lines of antibodies corresponding to the purification tags displayed on the phage. Anti-M13/HRP is flowed through the strip; afterwards, the test lines are treated with TMB. Development of one or more color test lines specifies the cancer types detected.

### EXAMPLE 12

A library of M13 phage particles displaying cyclic 20 amino acid peptides representing epitopes associated with anaplastic large cell lymphoma is prepared. The library contains 17 types of phage, and two million particles of this library are contacted with immobilized antibodies obtained from a patient suspected of relapse of this disease. After washing, the particles are introduced into RPA reactions with primers directed at the sequences encoding the peptides, as well as unrelated sequences for normalization and control. The early amplification of disease epitope sequences is taken as evidence of relapse of the disease.

### EXAMPLE 13

Nine wells of a polycarbonate PCR tube tray were incubated overnight at 4° C. with a twofold dilution series of anti-FLAG mAbs in PBS (500 ng, 250 ng, and 125 ng); each set of three wells contained 10 μL of one mAb dilution. Wells were then washed with 180 μL washing buffer (PBS+0.05% Tween 20) three times. Next, all wells were incubated for 2 hr at room temperature with 180 μL blocking buffer (PBS+1% nonfat milk). Wells were again washed with the washing buffer. Next, all wells received 9 μL WT MS2 VLP stock diluted in blocking buffer, where each 9 μL of this mixture contained 10,000 ng WT MS2 VLPs. Finally, each tenfold serial dilution of FLAG MS2 VLPs in PBS (1000 ng, 100 ng, and 10 ng) was added at 1 μL for each 3-well set that has been treated with the same mAb dilution for a total of 10 μL; incubation was carried out overnight at 4° C. After which, the wells were rewashed and the bound VLPs subjected to RT-qPCR.

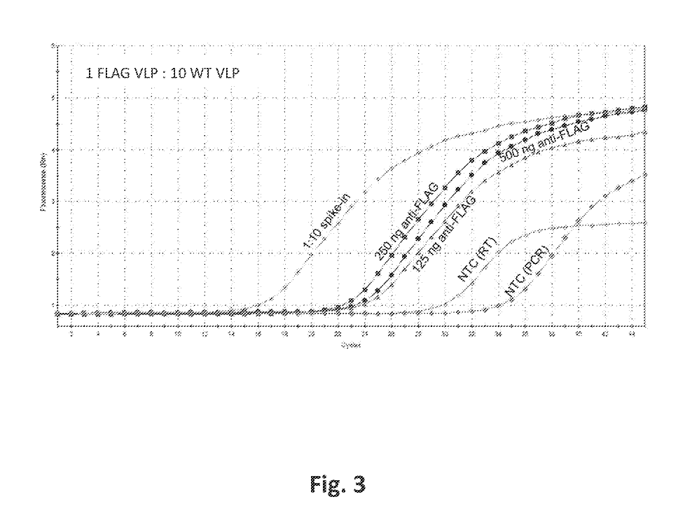

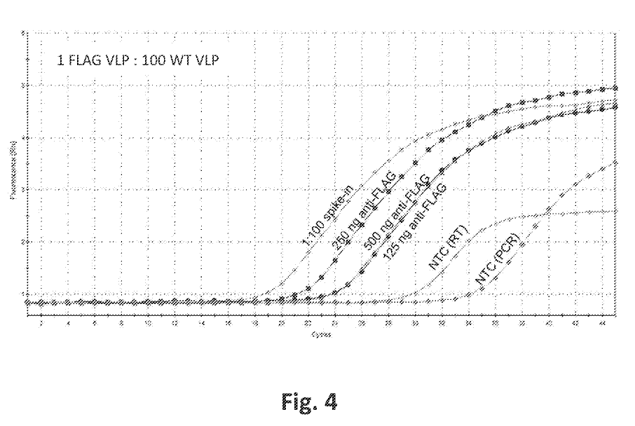

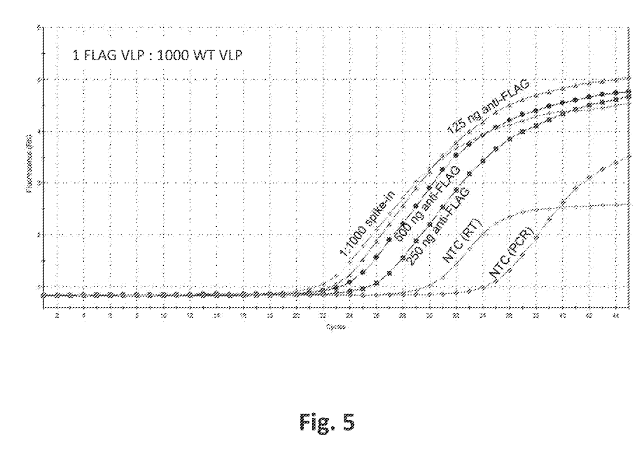

FLAG MS2 VLPs were captured against a background of WT MS2 VLPs supplied in excess (FIG. 3, FIG. 4, and FIG. 5). Based on ΔCts, roughly at least 1% of the FLAG MS2 VLPs were recovered from panning (Table 1). However, the highest recovery achieved in this group of runs was about 35% suggesting that higher recoveries are possible by further optimization of initial anti-FLAG MS2 mAb concentrations used for coating the well surface in addition to the quantity of MS2 VLPs offered.

TABLE 1
Estimated FLAG MS2 VLP recoveries based on threshold cycle
(Ct) values assuming 100% amplification efficiency.

| Run | Ct (dRn) | % recovery |
|---|---|---|
| 500 ng mAb/1:1000 spike-in | 21.91 | 19.08 |
| 250 ng mAb/1:1000 spike-in | 23.94 | 4.67 |
| 125 ng mAb/1:1000 spike-in | 21.03 | 35.11 |
| 1:1000 spike-in | 19.52 | 100.00 |
| 500 ng mAb/1:100 spike-in | 22.65 | 1.48 |
| 250 ng mAb/1:100 spike-in | 19.85 | 10.29 |
| 125 ng mAb/1:100 spike-in | 22.32 | 1.86 |
| 1:100 spike-in | 16.57 | 100.00 |
| 500 ng mAb/1:10 spike-in | 21.96 | 0.60 |
| 250 ng mAb/1:10 spike-in | 20.77 | 1.36 |
| 125 ng mAb/1:10 spike-in | 22.54 | 0.40 |
| 1:10 spike-in | 14.57 | 100.00 |

Without further elaboration, it is believed that one skilled in the art can, using the description herein, utilize the present invention to its fullest extent. The embodiments described herein are to be construed as illustrative and not as constraining the remainder of the disclosure in any way whatsoever. While the preferred embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A method of screening a biological sample for one or a plurality of diseases comprising:
obtaining a biological sample from a subject in need thereof, wherein the biological sample comprises one or a plurality of biomarkers, wherein each of the plurality of biomarkers is specific for at least one disease;
contacting the biological sample with a display library of peptides, wherein each peptide in the library may have a unique amino acid sequence, wherein each of the peptides is physically linked to a nucleic acid sequence that identifies or encodes it, and wherein at least one of the peptides is capable of binding to at least one of the biomarkers in the biological sample;
separating the bound peptide particles from the unbound peptide particle;
eluting the bound peptide particles from the bound state;
determining the identity of the nucleic acid sequences physically linked to the bound peptide particles; and
comparing the nucleic acid sequences of the bound peptide particles thus identified to a database of sequences representing a plurality of biomarkers of diseases.

2. The method of claim 1, wherein the disease is selected from breast cancer, prostate cancer, bladder cancer, soft tissue sarcoma, lymphomas, esophageal cancer, uterine cancer, bone cancer, adrenal gland cancer, lung cancer, thyroid cancer, colon cancer, glioma; liver cancer, pancreatic cancer, renal cancer, cervical cancer, testicular cancer, head and neck cancer, ovarian cancer, neuroblastoma, melanoma, acute lymphocytic leukemia, chronic lymphocytic leukemia, acute myeloid leukemia, ALCL, T cell leukemias, and B cell leukemias.

3. The method of claim 1 wherein the disease is a neurological disease.

4. The method of claim 1, wherein the neurological disease is selected from the group consisting of Alzheimer's disease (AD), pre-AD, Parkinson's disease, multiple sclerosis, fronto-temporal dementia, bipolar disorder, or schizophrenia.

5. The method of claim 1, wherein the method further comprises the step of immobilizing the plurality of biomarkers in the biological sample onto a support.

6. The method of claim 1, wherein the at least one biological sample comprises blood, dry blood, serum, plasma, saliva sample, mucosal swab, biopsy, a tissue, skin, hair, feces, urine, synovial fluid, cerebrospinal fluid, interstitial fluid, or pleural fluid.

7. The method of claim 1, wherein the plurality of biomarkers comprise immunoglobulin isotypes.

8. The method of claim 7, wherein the immunoglobulin isotypes comprise IgG, IgA, IgM, T cells, MHC, immune-fractionated antibodies, or combinations thereof.

9. The method of claim 7, wherein the method further comprises affinity purifying at least one immunoglobulin isotype from the biological sample.

10. The method of claim 9, wherein the affinity-purified immunoglobulin isotype is contacted with an immobilized display library of peptide particles.

11. The method of claim 1, wherein the pluralities of biomarkers comprise a plurality of proteins, and wherein expression of a unique combination of the plurality of proteins is specific for a specific disease.

12. The method of claim 1, wherein the display library of peptide particles comprises peptides displayed on a phage, spore, yeast, cell, ribosome, DNA, RNA, a colloid, a surface, or a label.

13. The method of claim 1, wherein the peptides are selected from the group consisting of a random peptide, a cyclic peptide, an enzymatically-modified peptide, a chemically-modified peptide, a glycosylated peptide, an entire protein, a random peptide, a moving-window peptide along a protein sequence, or a peptide representing an epitope specific for a disease marker, or a plurality of peptides representing immunodominant epitopes specific for a disease marker.

14. The method of claim 1, wherein the peptides are chemically or enzymatically modified.

15. The method of claim 1, wherein the display library of peptide particles is configured as a lateral-flow or as a microtiter plate assay.

16. The method of claim 1, wherein the method further comprises labeling sequences or chemicals associated with the displayed peptides.

17. The method of claims 16, wherein the labeling utilizes RNA, DNA, small organic molecules, fluorescence or metals.

18. The method of claim 1, wherein the display library of peptides comprises peptides encoded by the human genome.

19. The method of claim 1, wherein the display library of peptides comprises peptides representative of biomarkers for a plurality of cancers.

20. The method of claim 1, wherein the display library of peptides comprises peptides representative of biomarkers for a single disease.

21. A method for monitoring response to treatment or relapse of on-going disease in a subject in need thereof comprising:
obtaining a biological sample from the subject;
contacting the biological sample with a display library of peptides, wherein each peptide in the library may have a unique amino acid sequence, wherein each of the peptides is physically linked to a nucleic acid sequence that identifies or encodes it, and wherein at least one of the peptides is capable of binding to at least one biomarker in the biological sample;
separating the bound peptide particles from the unbound peptide particles;
eluting the bound peptide particles from the bound state;
determining the identity of the nucleic acid sequences physically linked to the bound peptide particles; and
comparing the nucleic acid sequences of the bound peptide particles thus obtained to a database of sequences representing a plurality of biomarkers for the disease, wherein the presence or absence of the at least one biomarker is indicative of the response to the treatment, or of relapse of the disease.

22. The method of claim 21, wherein the treatment is selected from a group consisting of chemotherapeutic agents, radiotherapeutic agent, cytokines, anti-angiogenic agents, apoptosis-inducing agents, immunotherapy, targeted therapy, cell based therapy, stem cell transplantation, bone marrow transplantation, and anti-cancer immunotoxins.

23. A method of establishing an immunosignature specific for diagnosing a disease in a subject in need thereof comprising:
obtaining a biological sample from the subject;
contacting the biological sample with a displayed peptide particle library, wherein each of the peptides has a unique amino acid sequence, wherein each of the peptides is physically linked to a nucleic acid sequence that identifies or codes it, wherein one or more of the peptides is capable of binding to at least one immunodominant epitope binding antibody specific for the disease present in the biological sample;
separating the bound peptides from the unbound peptides on the immobilized microarray display library;
eluting the bound peptides from the bound state;
determining the identity of the nucleic acid sequences physically linked to the bound peptide particles; and
comparing the nucleic acid sequences of the bound peptide particles thus obtained to a database of sequences representing a plurality of diseases to diagnose the disease, wherein the binding of the at least one antibody to a plurality of different peptides in the peptide array comprises an immunosignature specific for the disease.

24. The method of claim 23, wherein the biological sample comprises blood, dry blood, serum, plasma, synovial fluid, cerebrospinal fluid, interstitial fluid, or pleural fluid.

25. The method of claim 23, wherein the antibody in the biological sample comprises IgG, IgA, IgM, T cells, MHC, immune-fractionated antibodies, or combinations thereof.

26. The method of claim 23, wherein the method further comprises affinity purifying the at least one antibody from the biological sample.

27. The method of claim 26, wherein the affinity-purified antibody is contacted with an immobilized microarray display library of peptides.
